(19) United States
(12) Patent Application Publication
(10) Pub. No.: US 2010/0307945 A1
(43) Pub. Date: Dec. 9, 2010

(54) DEVICE FOR STORING DIAPERS

(76) Inventor: Claudia Rosa Halperin, Porto Alegre (BR)

Correspondence Address: JEROME D. JACKSON (JACKSON PATENT LAW OFFICE), 211 N. UNION STREET, SUITE 100, ALEXANDRIA, VA, 22314, US

(21) Appl. No.: 12/863,438

(22) PCT Filed: Jan. 20, 2009
(86) PCT No.: PCT/BR09/00020

§ 371 Date: Jul. 17, 2010

(30) Foreign Application Priority Data
Jan. 25, 2008 (BR) MU8800051-6

(51) Int. Cl. A61B 19/02

(52) U.S. Cl. 206/440

(57) ABSTRACT

Patent of Utility Model for a device that stores diapers of different sizes, in a deposit where both supply and removal of diapers (one at a time) is processed in a safe and practical way for users' personal hygiene. The use of the Diaper Holder starts when diapers are removed one by one, bottom-top wise. After a diaper has been removed from the bottom of the stack, it automatically lowers and the next diaper becomes available. Therefore, diapers are always ready to be removed, offering theirs users comfort and safety.

The present patent is a utility model of a constructive device introduced as a Diaper Holder. Its innovating conception based on significant technological and functional advances provides a range of real, extraordinary technical and practical advantages. In addition, users will find it very safe.

Infants wearing diapers, as well as adults, require special care all the time once diapers must be changed at daytime and/or nighttime. Due to the high number of changes, diapers must be handled carefully. This means they must be available immediately (one at a time), which involves controlling their quantity and facilitating their change. As a result, users feel safer.

In the market, one can see Diaper Holders where supply is done through the upper part and diapers are pulled out through a slot or hole in the lower part. These holders are usually wooden pieces where diapers are wrongly positioned (bent or stacked), which complicates their removal. Also, there are parts leaning against the change-maker (in the case of babies) or even some shelves to hold disposable diapers. All in all, this system lacks hygienic conditions (diapers are exposed) and may cause skin irritation. Diaper Holders, in the form of bags hanging from the change-maker are also found.

Based on analyses of the pieces available to users, one can observe how difficult it is to use them and how risky it might be for people who need them, thus reinforcing the belief that the technique currently employed is rather inefficient and sometimes hazardous.

In the baglike type, the babysitter (infant diapers) must often use her both hands to reach the diapers and sometimes two come out at a time. As a result, the baby is left unattended and might fall off; in other words, the infant's safety is at a risk. This kind of accident has been widely reported by pediatricians and hospitals owing to its high rate.

Another problem some products present is related to their design/shape. They are made from unsuitable and excessively hard material with straight angles (corners). For instance, wood might expose an infant or adult to impacts and therefore to lesions caused by the apparatus.

In addition, there are Diaper Holders that do not allow the user to control the number of diapers available, for example, Patent N. W02006071320A1. In practice, the caretaker leaves the baby alone in the critical hour if there are no diapers available at that time. Adults, in this case, must be assisted by another person.

Also, there are Diaper Holders in the market that are attached to the change-makers, usually consisting of heavy pieces, easily accessed by infants who move their arms and legs constantly. The problem is, these holders take too much space, thus directly interfering with the infant's comfort and safety.

For the purpose of storing adult diapers, no specific model was found. Therefore, this product, object of the present patent, directly and immediately supplies this need for adult diapers in hospitals, old people's home and asylums.

In view of these problems and aiming to overcome them, a device for storing diapers was devised, object of the present patent, which consists of a dispenser (a box) that offers its users practicality, hygiene and above all, safety.

The way the device for storing diapers was built, out of plastic material, preferably used on the wall, was based exactly on the technical problems presented by similar products. However, it is different in that users find it comfortable and safe.

The pictures herein attached show the layout of the device for storing diapers, object of the present patent, in which:

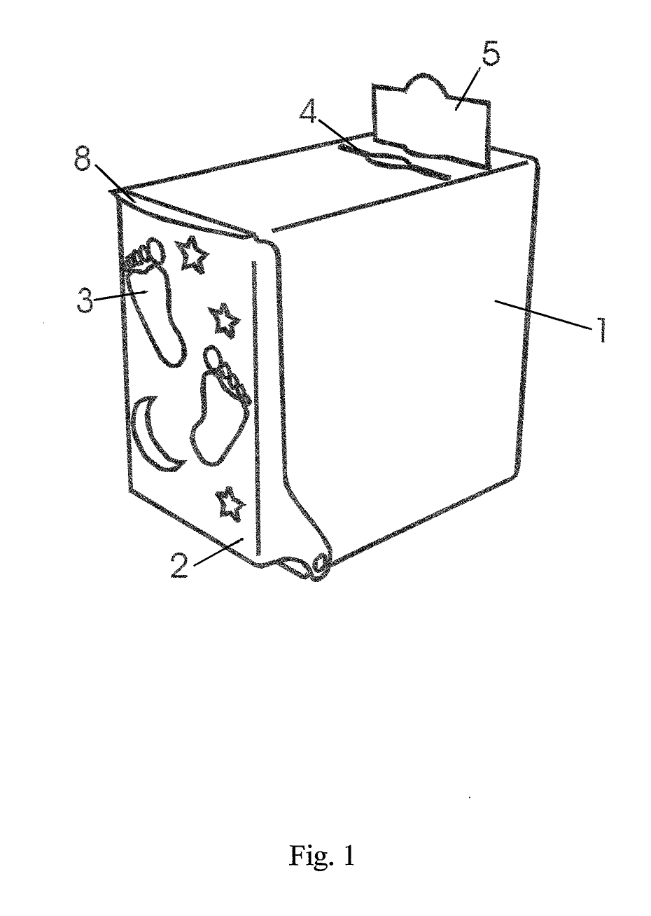

FIG. 1 shows it in perspective (the front opening closed);

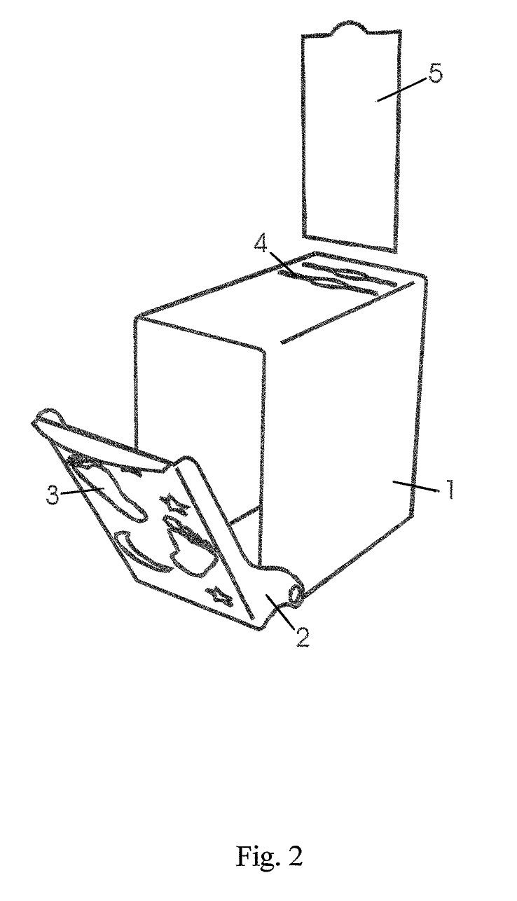

FIG. 2 shows it in perspective (the front opening opened);

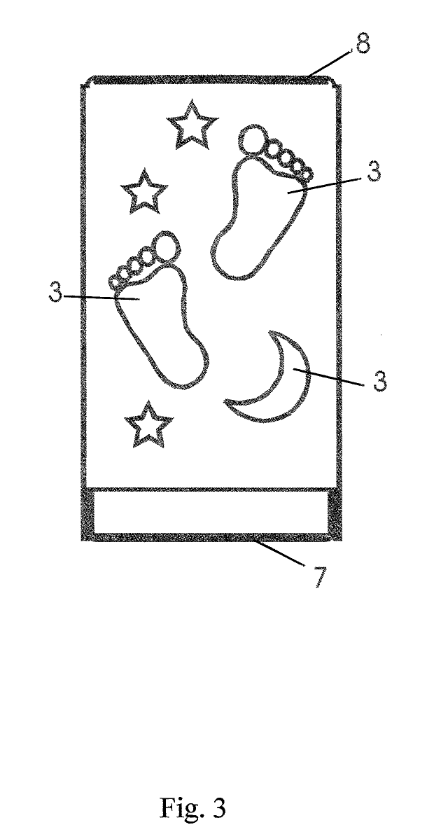

FIG. 3 shows the front view;

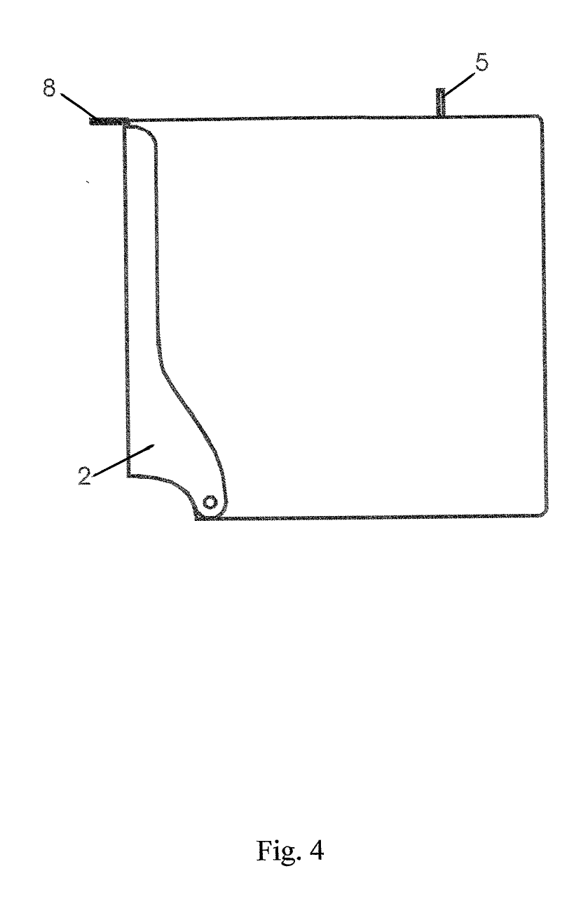

FIG. 4 shows the right side view;

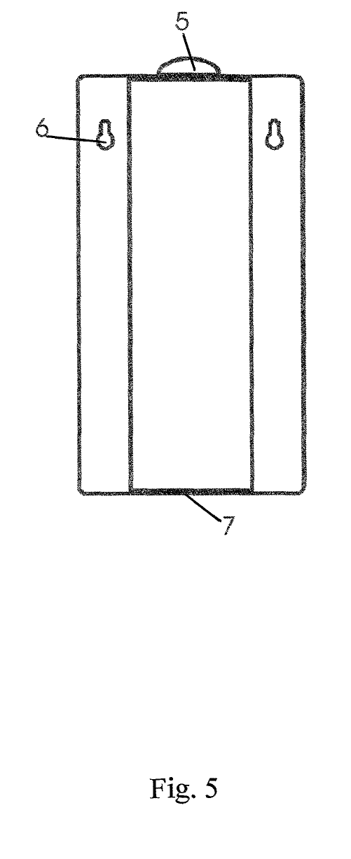

FIG. 5 shows the backside view (with clamps);

Based on the pictures above, the Diaper Holder model, object of the present patent, consists of a dispenser, a deposit (1) between 12.0 and 30.0 cm wide, 12.0 and 30.0 cm deep and 5.0 to 90 cm high, closed by an articulated door (2) with displays (3), two upper openings (4) that steer a plate (5) for regulation according to the size of the diapers, comprising all sizes, from Newborns to Extra Large, as well as S-M-L-XL for adults. (And other nomenclatures that determine the size of the diapers). It is preferably fixed on the wall by means of hollowed elements (6), positioned in the backside of the device or other fixation elements. It can also be used on the table, where it is completely settled (7).

Diapers are supplied through the front door opening (2) of the Diaper Holder, which has a kind of handle (8). Therefore, diapers can be placed one on top of the other in a stack positioned lengthwise. During supply, the door remains open but does not separate from the dispenser, facilitating its closure right after the process is through.

The correct position of the internal plate (5) is also essential since it provides stability to all diaper sizes the Diaper Holder contains, because not all sizes are likely to be used and they may vary according to brands and formats utilized.

Once the supply has been correctly performed, the door is closed and the quantity of available diapers is shown both through the displays and/or hollowed elements and the lower part where the first diapers are to be removed, because the use of the Diaper Holder starts when the diapers are removed one-by-one from the bottom and upwards. After removing the diaper from the part of the stack, automatically the stack lowers and the next diaper becomes available. The diapers are then always ready to be removed, offering their users comfort and safety.

1. Device for storing diapers characterized by a deposit, closed by an articulated door, with displays and/or hollowed elements, having two upper openings that steer a plate for regulation according to the size of the diapers, comprising all sizes, from Newborns to Extra Large, as well as S-M-L-XL sizes and other nomenclature determining the size of diapers, preferably fixed on the wall by means of hollowed elements positioned on the backside of the apparatus or through other means, so that the device may be utilized by users in the moment of personal hygiene, fully meeting their needs.

2. Device for storing diapers based on claim 1 above, characterized by an articulated door with displays and/or hollowed elements aiming to keep track of the quantity of diapers available for immediate use through the transparency the sketches provide.

3. Device for storing diapers based on claim 2 above, characterized by an articulated door that, when open, presents the device in a position allowing the supply of diapers after being correctly closed, thus dispensing the first diaper to be removed from the stack through the lower part of the deposit.

4. Device for storing diapers based on claim 1 above, characterized by a regulation system for the different sizes of diapers, a mobile plate being positioned according to the size of the diaper so as to avoid that the small diapers become loose in the deposit, the large diapers fitting in the deposit.

5. Device for storing diapers based on claim 1 above, characterized by a fixation system, preferably on the wall, on the backside of the piece in order to enhance the safety of users, mainly infants.
